(19) United States
(12) Patent Application Publication
(10) Pub. No.: US 2021/0393667 A1
(43) Pub. Date: Dec. 23, 2021

(54) TARGET FOR ANTI-CANCER THERAPY

(71) Applicant: Phoremost Limited, Cambridge (GB)

(72) Inventors: Grahame James MCKENZIE, Cambridge (GB); Samuel Peter BARKER, Cambridge (GB)

(73) Assignee: Phoremost Limited, Cambridge (GB)

(21) Appl. No.: 17/285,598

(22) PCT Filed: Oct. 16, 2019
(86) PCT No.: PCT/GB2019/052954

§ 371 Date: Apr. 15, 2021

(30) Foreign Application Priority Data
Oct. 16, 2018 (GB) 1816825.2

(51) Int. Cl. A61K 31/7076; A61K 31/44; A61P 35/00; A61K 45/06

(52) U.S. Cl. CPC A61K 31/7076; A61K 45/06; A61P 35/00; A61K 31/44

(57) ABSTRACT

The present invention relates to a composition and uses thereof. The invention relates to a composition for use in stimulating neoantigen production in a cancerous tumour. In particular, the composition comprises a compound or a pharmaceutically acceptable salt thereof, wherein the composition is a negative modulator of the expression, function or stability of ribonucleotide reductase.

This invention relates to the identification of RNR as a target for therapeutic formulations for increasing the efficacy of immune checkpoint regulators in the treatment of certain cancers.

### BACKGROUND OF THE INVENTION

Immune checkpoint regulators (ICR) show enormous promise in the treatment of cancer. However, to date, the benefits of ICR treatment is generally limited to those tumours having inherent genomic instability such as Microsatellite Instable (MSI) tumours, representing less than 5% of tumours. There is therefore a need to find new mechanisms for improving ICR response in those tumours that do not naturally show genomic instability.

### SUMMARY OF THE INVENTION

The present invention relates to the identification of ribonucleotide reductase (RNR), and its component genes or their protein products, as a target whose inactivation or modulation leads to an increase in mutational rates, as assessed, for example, by a frameshift reporter assay. Stimulating an increase in the mutational rate in a tumour leads to an increase in neoantigen expression. Thus RNR is a target for the treatment of cancer. In particular, inactivators or modulators of RNR may make cancer cells more susceptible to immunotherapy.

Accordingly, in a first aspect, the invention provides a composition for use in stimulating neoantigen production in a cancerous tumour in a subject, wherein the composition is for improving the subject's immune response against the cancerous tumour, and wherein the composition comprises a compound or a pharmaceutically acceptable salt thereof, which is a negative modulator of the expression, function or stability of ribonucleotide reductase (RNR).

In one embodiment, the compound is a negative modulator which causes frameshift mutations as measured by a frameshift reporter assay. Suitably, the frameshift reporter assay measures a change in luminescence through a reporter assay as described herein wherein the compound for use in accordance with the invention negatively modulates the expression, function or stability of RNR and results in a mean luminescence greater than 3 standard deviations from a control in a frameshift reporter assay as described herein, or wherein inhibition results in a mean luminescence which is more than a 150% increase compared to a control.

In one embodiment, the composition in accordance with the invention comprises a compound having a therapeutic concentration range wherein frameshift mutations increase at a greater rate than cell viability decreases. Suitably frameshift mutations are measured by a frameshift reporter assay, while cell viability is measured using a standard cell viability assay. In another embodiment, the compound has a Kᵢ (RNR) equal to or below its GI₅₀. Suitably, Kᵢ (RNR) may be measured by application of the Michaelis-Menten model; GI₅₀ may be measured in standard assays such as the assay described herein, for example.

In another embodiment, there is provided a composition in accordance with the invention which is capable of delivering a concentration of compound to the cancerous tumour, wherein the concentration of compound is within the therapeutic concentration range. Suitably the composition comprises a compound at a suitable concentration range to increase frameshift mutations at a greater rate than decreasing cell viability and thereby have a therapeutic effect.

In one embodiment, the composition in accordance with the invention comprises a compound which is an inhibitor of ribonucleotide reductase. Suitably, such a compound may be a polypeptide, a polynucleotide, an antibody, an aptamer, a peptide, a small molecule, an RNA-based drug, such as siRNA or RNAi, a genetic construct for targeted gene editing, or any other suitable chemical.

Suitably, the composition in accordance with the invention comprises a small molecule inhibitor of RNR, such as any of clofarabine, triapine, cytarabine, cladribine, azathioprine, fludarabine, 5-flurouracil, hydroxyurea, motexafin gadolinium, gallium maltolate and gallium nitrate, or a pharmaceutically acceptable salt thereof. In one embodiment, the composition in accordance with the invention comprises a compound selected from cladribine, clofarabine, triapine, cytarabine and fludarabine, or a pharmaceutically acceptable salt thereof.

In one embodiment, of any aspect of the invention, the cancerous tumour is microsatellite stable. In other embodiments, the cancerous tumour is one which is otherwise unlikely to be responsive to treatment with immune checkpoint regulators (ICR). Suitably such cancerous tumours may be characterised by a low mutator phenotype.

In one embodiment, the cancerous tumour is selected from neuroblastoma, glioma, glioblastoma, prostate, ovary, myeloma, pancreas, breast, papillary kidney, B cell lymphoma, clear cell kidney, head and neck, liver, cervix, uterus, bladder, colorectal, small cell lung, espohagus, stomach, non-small cell lung cancer, and melanoma.

In one embodiment there is provided a composition in accordance with the invention wherein the treatment comprises administering a therapeutically effective amount of the compound to the subject.

In another embodiment, there is provided a composition in accordance with the invention for use in combination with an immunotherapy, suitably an immune checkpoint inhibitor or a combination of immune checkpoint inhibitors. An immune checkpoint inhibitor may be selected from PD-1 inhibitor, a PD-L1 inhibitor, and a CTLA-4 inhibitor. Suitable immune checkpoint inhibitors include pembrolizumab, nivolumab, ipilimumab, atezolizumab, avelumab, durvalumab, tremelimumab, tislelizumab, pidilizumab, AMP-224, AMP-514, PDR001, BMS-936559.

In one embodiment, the immunotherapy is administered subsequent to or concurrent with the composition in accordance with the invention.

In another aspect, the invention provides a method for improving the immune response to a cancerous tumour in a subject in need thereof, the method comprising the steps of:
a. assessing the level of neoantigen presentation of the cancerous tumour;
b. determining whether the level of neoantigen presentation is below a threshold to induce an immune response, and
c. if the level of neoantigen presentation is below a threshold to induce an immune response, administering a composition comprising a compound or a pharmaceutically acceptable salt thereof, which is a negative modulator of the expression, function or stability of ribonucleotide reductase.

In one embodiment, the level of neoantigen presentation is assessed with reference to the number of mutations in the genome of the cancerous tumour. In some embodiments, the threshold to induce an immune response may be assessed as a significant increase in mutations over baseline. This may vary depending on tumour type. Suitably, the threshold to induce an immune response is approximately 90 to 150 mutations per megabase, preferably 100 mutations per megabase.

In one embodiment, the method in accordance with this aspect of the invention comprises the further steps of:
d. reassessing the level of neoantigen presentation of the cancerous tumour; and/or
e. Administering an immunotherapy to the subject.

Suitably, the composition for administration is a composition in accordance with any aspect or embodiment of the invention.

### DETAILED DESCRIPTION OF THE INVENTION

### RNR

Ribonucleotide Reductase (RNR), is also known as ribonucleoside diphosphate reductase (rNDP). RNR is made up of two subunits, large RNR1 and small RNR2. These subunits associate to form an active heterodimeric tetramer. In humans, the RNR1 subunit is encoded by the RRM1 gene while there are two isoforms of the RNR2 subunit, encoded by the RRM2 (Entrez Gene ID: 6241; NM_001034.3 at chr2:10122736-10131419) and RRM2B genes. RRM2 encodes the catalytic domain and therefore is likely to be the main target for modulators/inhibitors.

RNR catalyzes the formation of deoxyribonucleotides from ribonucleotides. As described herein, siRNA corresponding to Ribonucleotide-Diphosphate Reductase subunit M2 (RRM2) has been shown to induce an increase in mutations as measured in an FSR assay. Accordingly, in some embodiments, a negative modulator of the expression, function or stability of ribonucleotide reductase is a modulator which modulates the activity of RNR2 subunit encoded by RRM2 and RRM2B genes. In other embodiments, a negative modulator may interact with the RNR1 subunit of RNR.

During cell division and DNA repair, RNR regulates the total rate of DNA synthesis to maintain the DNA to cell mass at a constant ratio. Ribonucleotide Reductase (RNR) is preferentially expressed in rapidly dividing cell types such as cancers and the haematopoietic stem cell compartment, which provides the therapeutic window for its clinical deployment. Loss of RNR causes abnormal DNA metabolism precursor pools, notably a lack of dNTPs, leading to increased rates of spontaneous mutagenesis (Mathews; Nat. Rev. Cancer, 2015, September; 15(9): 529-39; PMID: 26299592). This suggests an explanation for the effects seen in the FSR assay described herein. High expression of RNR has been positively associated with a poor survival prognosis, suggesting an increased reliance of cancer cells upon the target's function.

### Methods for Detecting Increased Mutations

By “stimulating neoantigen production” it is meant that a composition in accordance with the invention is one which increases mutations in a cell compared to a control composition. Methods for measuring increases in mutation, such as an increased mutational load are known to those skilled in the art. Suitable assays for the effect of a composition include the Frame Shift Reporter (FSR) assay as described herein, as well as other assays familiar to those skilled in the art (see, for example, Parsons et al. Cell. 1993 Dec. 17; 75(6):1227-36). The example of a frameshift reporter assay described herein includes using a CA tract (CA₍ₙ₎), such as CA₍₁₈₎, or CA₍₂₀₎, for example, upstream of a reporter coding sequence such as a luminescent reporter enzyme, for example, NanoLuc® (Nluc). The CA tract renders the reporter enzyme coding sequence out of frame. When the regulation of frameshift mutations is modulated or inhibited by modulating or inhibiting a target gene, frameshift mutations occur leading to expression of the reporter enzyme resulting in a detectable luminescent signal in the presence of the corresponding luciferase substrate reagent. Such an increase in mutational rates or loads can equate to an increase in dynamic mutational loads, or to an increase in neoantigen creation.

Suitably a “simple nucleotide sequence” may be any genomic repeat sequence which is known to be a site for replicative errors, for example, one that is known to accumulate mismatches during DNA replication. Suitable genomic repeat sequences include those sequences which are identified as a microsatellite region such as, for example, a microsatellite repeat or a sequence which is associated or indicative of a replicative repair deficiency. Suitable such sequences include poly A sequences such as A₍₁₇₎. In another embodiment, a dinucleotide repeat sequence may be used such as CA or GT, in particular CA₍ₙ₎ where n may be any number. In one embodiment, the dinucleotide repeat sequence is CA₍₁₄₎, CA₍₁₈₎ or CA₍₂₀₎, also referred to as a CA₍ₙ₎ repeat “tract” sequence. Other repeat sequences such as trinucleotide repeats are also envisaged.

Suitably the reporter coding sequence is a nucleic acid sequence which encodes a reporter moiety. Suitable reporter moieties will be familiar to those skilled in the art and include selectable markers. Examples of reporter moieties include beta-galactosidase, a luciferase such as NanoLuc®, reporters which can be used to generate a fluorescent signal and so forth. Advantageously, the reporter moiety is one which has a large and linear dynamic range such that a small change in the number of in-frame reporter moieties expressed results in a positive signal, thus allowing a sensitive assay. Suitable selectable markers will be familiar to those skilled in the art and include antibiotic resistance genes and drug selection markers such those genes encoding resistance to antibiotics such as puromycin, G418, hygromycin, blasticidin, puromycin, zeocin or neomycin, for example. Advantageously using a selectable marker allows a survival signal to be detected i.e. only those cells which a test compound acts as a modifier of a DNA repair or nucleic acid editing gene, or its protein product will survive when grown in the presence of an antibiotic.

Accordingly, a composition or compound for use in stimulating neoantigen production can be identified in a suitable screening method or assay. Suitably the cells for use in a screening method or assay in accordance with the invention are a mammalian cell line. Suitable mammalian cell lines include HEK293 cells such as HEK293A, FT or T cells although other cell lines are envisaged.

In one embodiment, an increased signal from a reporter construct is identified as an increased read-out from the reporter construct in the presence of the test compound compared to the read-out in the absence of the test compound.

In further embodiments, a composition or test compound may be a candidate anti-cancer compound for use in accordance with the invention because it is effective to act as a negative modulator of the expression, function or stability of RNR. Thus a composition may be effective to either reduce or modify expression of a RNR or to act as an inhibitor or modifier of the protein product of an RNR gene so as to inhibit or alter DNA repair activity, or otherwise dynamically generate an increased cellular mutation burden. Examples of suitable methods for measuring the rate of DNA mutation are known to those skilled in the art. DNA mutation may be measured as a number of mutations/megabase (Mb) of DNA. For example, functional inactivation of RNR gene or enzyme may be determined by sequencing repetitive DNA elements or cDNAs. Exome sequencing of cells treated with a test compound compared with untreated cells may be used to measure mutational loads. For example, exome sequencing from cells collected longitudinally at distinct time-points can be performed. Importantly, an increased rate of DNA mutation or cDNA epimutation not only leads to an increase in the number of mutations and antigens but also to the acquisition of new mutations over time as a result of DNA repair inactivation or modification or nucleic acid editing modification. This leads to a dynamic hypermutation state. The mutation (and therefore the corresponding neo-antigen) profiles therefore preferably dynamically evolve over time such that the genomic landscape rapidly and dynamically evolves with the continuous emergence of neo-antigens.

In one embodiment, stimulation of neoantigen production may be measured as a high mutational load observed in treated cells, thus indicating that a composition or test compound is a candidate anti-cancer agent. Suitably, a high mutational load may be expressed as a mutation rate wherein an increased rate of DNA mutation is in the region of 10-100 mutations/megabase of DNA. In another embodiment, an increased mutational burden may be in the region of over 100 mutations/megabase of DNA.

Suitably a “control” is a control sample from which an inhibitor is absent. Where the inhibitor is an RNA-based inhibitor such as an siRNA molecule, a control is a sample which has been treated with a “Non-Targeting Control” (NTC) which may be a combination of siRNAs which are non-gene targeting e.g. a random sequence.

RNAseq analysis may also be used to identify the proportion of mutated genes that are transcribed and therefore can act as neo-antigens. Microsatellite instability assays may also be used.

In one embodiment of a method for screening in accordance with the invention, cells expressing RNR can be a human tumour cell line.

### Inhibitors

The term “modulator” refers to a compound which changes the activity of a gene, or its protein product, in the presence of that compound compared to the activity in the absence of that compound. In the context of the present invention, a “modulator” is preferably a negative modulator which can be an inactivator/inhibitor of the gene or its protein product. For example an inactivator or inhibitor may be one which reduces gene activity through inhibiting the enzymatic activity of the protein encoded by the gene, for example.

A modulator, as defined above, is a compound which works to modify a component either by acting at the gene, RNA or protein level. In particular, references to a “gene” as described herein are to the gene per se as well as the protein encoded by the gene (i.e. its protein product). Methods for determining whether a compound is a modulator of a gene/protein, such as RNR, include methods for detecting binding to a particular gene of interest, functional assays for a particular gene/protein which will be familiar to those skilled in the art and methods for detecting a defect in a gene/protein through measuring an increase in mutations, such as frameshift mutations. Assays for determining RNR activity are described, for example, in Zhou et al. (Cancer research. 2013; 73(21):10.1158/0008-5472.CAN-13-1094. doi:10.1158/0008-5472.CAN-13-1094).

In one embodiment of any aspect of the invention, a modulator, such as an inhibitor of RNR may be a molecule which provides modulation, e.g. inhibition, through altering the gene at the level of modifying expression of the gene by altering its genetic code. Suitable methods for modifying gene expression are known to those skilled in the art and include using genome editing methods. For example, a gene may be knocked-out, silenced or modulated using a CRISPR-based or other genome editing approach. Suitable methods for genome editing are familiar to those skilled in the art. Other methods for knocking out or modifying gene expression from a particular gene include using an interfering RNA approach e.g. siRNA. Other types of modulators, such as inhibitors, are also described herein.

Suitably, modulation, such as inhibition, of a “frameshift-regulating” gene will result in the DNA mutation or RNA epimutation profiles dynamically evolving over time, and therefore correspondingly alter the expression and presentation of neo-antigens to the immune system. In one embodiment, modulation leads to the generation of neo-antigens (tumour antigens). In one embodiment, the invention relates to increase of ‘dynamic’ mutational loads that can be achieved by inactivation of specific genes/proteins.

In another embodiment, modulation, such as inhibition, of a “frameshift-regulating” gene may be determined by measuring DNA or RNA mutations and, in particular by measuring the rate of mutation. Such mutations may include point mutations, frameshift mutations and mutations as a result of homologous recombination. Suitable methods for determining an accumulation of mutations or rate of mutation are described herein. In one embodiment, increased mutation rates may be determined by measuring the number of neo antigens.

### Small Molecule Inhibitors

Small molecule inhibitors of the present invention have a molecular weight of up to 900 daltons, and preferably up to 500 daltons. In one embodiment, the modulator may be a small molecule such as an “anti-metabolite” small molecule i.e. those small molecules which inhibit dNTP production and are incorporated into DNA instead of the natural bases. “Anti-metabolite” small molecules include “nucleoside anti-metabolites” such as “purine nucleoside analogs”. Suitable such purine nucleoside analogs include those small molecules which are currently marketed products such as clofarabine.

Clofarabine ((2R,3R,4S,5R)-5-(6-amino-2-chloropurin-9-yl)-4-fluoro-2-(hydroxymethyl)oxolan-3-ol), “Clolar” (US and Canada) and “Evoltra” (Europe and AU/NZ), “Clofarex”), is a second generation purine nucleoside analog with antineoplastic activity (reviewed, for example in “Chemotherapy for Leukemia: Novel Drugs and Treatment (pp. 261-286; DOI 10.1007/978-981-10-3332-2_16); described in U.S. Pat. No. 5,034,518). Clofarabine is phosphorylated intracellularly to the cytotoxic active 5-triphosphate metabolite, which inhibits the enzymatic activities of ribonucleotide reductase and DNA polymerase, resulting in inhibition of DNA repair and synthesis of DNA and RNA. This nucleoside analog also disrupts mitochondrial function and membrane integrity, resulting in the release of pre-apoptotic factors, including cytochrome C and apoptotic-inducing factors, which activate apoptosis.

The cytotoxic activity of clofarabine is due to both its inhibition of ribonucleotide reductase and its efficient incorporation in DNA, where it inhibits DNA synthesis. While some activity has been observed in lymphoid malignancies, clinical efficacy has primarily been observed in acute leukemias.

The recommended dose of clofarabine for one of the currently approved indications of adult acute leukemia is 40 mg/m2/day×5 days which results in plasma levels of around 1 μM. This dose provides for a cytotoxic effect wherein the leukaemia cells are killed by the drug.

Other “Anti-metabolite” small molecules include gemcitabine (Gemzar), triapine, cytarabine (ara-C, Cytosar-U), Cladribine (Leustatin), Azathioprine (Azasan, Imuran), Fludarabine (Fludara), 5-Fluorouracil (Adrucil), hydroxyurea, motexafin gadolinium, gallium maltolate and gallium nitrate.

In one embodiment, in accordance with the present invention, an inhibitor of RNR is provided at a dose which allows for the accumulation of mutations in a cancerous cell, whilst not being cytotoxic. Suitably, such a dose may be below the dose at which a cytotoxic effect may be achieved.

### Treatment and Diagnosis

Suitably in a treatment in accordance with the invention, a modulator, such as a negative modulator, inhibitor, or combination is provided in a therapeutically effective amount. The term “therapeutically effective amount” refers to that amount of the compound being administered which will relieve to some extent one or more of the symptoms of the disease or disorder being treated. In one embodiment, the treatment may be relatively prolonged, e.g. over a number of months. In the context of the present invention, a “therapeutically effective amount” is one which increases the appearance of neoantigens in a patient's tumour cells whilst not being cytotoxic to said cells such that the neoantigen presenting cells are maintained long enough to be recognised by an immune response.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder. In the context of a cancer treatment, “treating” may include a reduction in the number of cancerous cells in a subject may be determined by detecting a reduction in tumour mass or size. A reduction in the number of cancerous cells may also be determined by any clinical endpoint which indicates a successful cancer therapy e.g. an absence of tumour relapse or recurrence or an increase in survival rate compared to the average survival rate observed in similar individuals in the absence of said treatment. In some embodiments, the present invention provides a treatment which improves the subject's immune response against the cancerous tumour. In some embodiments, an improved immune response may be quantified by quantifying an increased intratumoral penetration of activated CD8⁺ T cells.

In one aspect, the invention provides a method for the treatment of a cancerous tumour in a subject in need thereof comprising administering a composition comprising a compound which is a negative modulator or inhibitor of the expression, function or stability of ribonucleotide reductase wherein the compound stimulates neoantigen production in the cancerous tumour. Suitably the compound is one which results in frameshift mutations as measured by a frameshift reporter assay/increases neoantigen levels in a subject]. In one embodiment, the compound is an inhibitor or RRM2 gene or its protein product. Suitably, the compound is an anti-metabolite small molecule such as clofarabine.

In another aspect, there is provided a method for treating cancer comprising:
a) providing i) a subject having cancerous cells, and ii) a negative modulator, suitably an inhibitor of an RNR complex gene, or its protein product, whose modulation, suitably inhibition, leads to frameshift mutations; and
b) treating said subject with said modulator, suitably said inhibitor, wherein said treating reduces the number of cancerous cells in said subject.

Prior to administration of a modulator as part of a treatment in accordance with the invention, a patient may be screened to determine whether a cancer from which the patient is or may be suffering is one which is characterised by the presence of an active form of a particular gene such as a “frameshift-regulating” gene as described herein or an enzyme encoded by such a gene. For example, a cancer may be identified as an MMR+ve cancer i.e. a cancer in which those genes involved in MMR are active and/or present or have not been lost as part of the tumour evolution. Presence of genes may be detected using methods familiar to those skilled in the art and include, for example, PCR methods. In some aspects or embodiments, a patient is identified as one with a Tumour Mutation Burden (TMB) below a certain threshold. Tumour Mutation Burden (TMB) is the measurement of the number of mutations carried by tumour cells. TMB is a surrogate measure for the number of expressed neoantigens and can therefore be used as a biomarker to predict response to immune checkpoint therapy (Osipov et al., 2019; Schrock et al., 2019; Goodman et al., 2019; Peters et al., 2019; Ricciuti et al., 2019; Hanna et al., 2018). Studies have reported that a higher TMB generates an increased level of neoantigens. A study investigated the contribution of single nucleotide variant (SNV) and insertion-deletion derived frame-shift (FS) mutations to tumour neoantigen expression and reported that in comparison to SNVs, FS mutations generate more and potentially more immunogenic neoantigens per mutation (Turajlic, Samra et al. The Lancet Oncology, 2017 18(8): 1009-1021). Furthermore, studies that have focused specifically on tumour FS mutation burden in the context of immune checkpoint therapy report a significant correlation between FS burden and an improved treatment response in melanoma, renal, head and neck, and lung cancers (Turajilic et al., 2017; Hanna et al., 2018; Chae et al., 2019).

In other aspects of the invention, there is provided a modulator, such as an inhibitor, of a “frameshift-regulating” gene as described herein in the manufacture of a medicament for use in the treatment of cancer. Suitable cancers are described herein and include cancers otherwise having a low mutator phenotype.

In another aspect, the invention provides an in vitro method for diagnosing a cancer which is suitable for treatment by immunotherapy comprising:
a) taking a sample of said tumour,
b) analysing said sample to determine the presence or absence of protein or mRNA sequence of an RNR complex gene, including RRM2;
c) comparing the sequence of said gene in a tumour sample with the sequence in a non-tumour sample

wherein a defect in the sequence of an RNR complex gene in the tumour sample compared to the sequence of said gene in a non-tumour sample is indicative that said patient has a tumour suitable for treatment by immunotherapy.

In one embodiment, a mutation in an RNR complex gene is detected. Such a “mutation” may be a whole or partial deletion of the “frameshift-regulating” gene or a point mutation to render it inactive.

Examples of cancers (cancerous tumours) (and their benign counterparts) which may be treated include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the oesophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukaemias, lymphomas) and premalignant haematological disorders and disorders of borderline malignancy including haematological malignancies and related conditions of lymphoid lineage (for example acute lymphocytic leukaemia [ALL], chronic lymphocytic leukaemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenous leukaemia [AML], chronic myelogenous leukaemia [CML], chronic myelomonocytic leukaemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocytic leukaemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcoma protuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum). Those subsets of any of these cancers having a low mutator phenotype/low TMB may be preferred. In one embodiment, the cancerous tumour for treatment is a microsatellite stable tumour.

In one embodiment, a tumour for treatment in accordance with the invention may be one which has a mutation in a DNA repair or nucleic acid editing gene.

For example, Hereditary Non Polyposis Colon Cancer (HNPCC) is a hereditary cancer syndrome comprising a germline mutation in genes controlling MMR. Accordingly, HNPCC is one cancer syndrome that may be treated in accordance with the invention or using a compound identified using a method of screening in accordance with the invention. Other suitable cancers having mutations in DNA repair genes will be familiar to those skilled in the art.

Other cancers that have alterations in MMR genes are described, for example, in Xiao et al. (2014) and Okuda et al. (2010), and include sporadic colorectal cancers, ovarian and endometrial cancers.

Suitably, the subject having cancerous cells i.e. the subject to be given the cancer treatment is a subject which has a tumour which is proficient in MMR, DNA repair or nucleic acid editing i.e. it is not a tumour in which a mutation in a gene to be inhibited in accordance with the invention has already been identified. Methods for identifying a mutation in in a tumour sample will be familiar to those skilled in the art and are described herein.

In particular, the subject would not already have a deficiency in the RNR gene.

In one embodiment, the invention provides a use, method or a combination in accordance with any aspect of the invention wherein the cancer is any cancer which has a low mutator phenotype.

In one embodiment, a diagnostic test may be undertaken. Suitably, a biological sample taken from a patient may be analysed to determine whether a cancer, that the patient is or may be suffering from, is one which is characterised by a genetic abnormality or abnormal protein expression which may make it particularly susceptible to a use of an inhibitor in accordance with the invention. The diagnostic tests are typically conducted on a biological sample selected from tumour biopsy samples, blood samples (isolation and enrichment of shed tumour cells), stool biopsies, sputum, chromosome analysis, pleural fluid and peritoneal fluid.

### Compositions and Combinations

In one embodiment, where the modulator is for use in combination with a different cancer treatment, that cancer treatment may be an immunotherapy i.e. a therapy that uses the immune system to treat cancer. A wide range of immunotherapy approaches will be known to those skilled in the art. In particular, compounds (e.g. peptides, antibodies, small molecules and so forth) that act as immune checkpoints, i.e. affect immune system functioning may be used in combination with a use or method of treatment in accordance with the invention. For example, an immune checkpoint therapy (such as an immune checkpoint regulator or inhibitor) may block inhibitory checkpoints so as to restore immune system function. Suitable targets for compounds that act on immune checkpoints include, for example, programmed cell death 1 protein (PDCD1, PD-1; also known as CD279) and its ligand, PD-1 ligand 1 (PD-L1, CD274). PD-L1 plays a key regulatory role on T cell activity and cancer-mediated upregulation of PD-L1 on the cell surface has been observed to inhibit T cells which might otherwise attack a cancer cell. Therapeutic antibodies have been developed to bind to either PD-1 or PD-L1 to allow T-cells to attack the tumour by blocking this inhibitory action. Suitable compounds for use in combination with a method of treatment in accordance with the invention therefore include therapeutic antibodies which inhibit PD-1 pathways such as an anti-PD1 antibody (e.g. Nivolumab, Pembrolizumab) and anti-PDL-1 antibodies. Other antibodies include those targeting CTLA-4 e.g. anti-CTLA-4 antibodies. Other immune checkpoint therapies may be developed to similar immune checkpoint targets. In one embodiment the immunotherapy for use in combination with the inhibitor may be a combination of molecules targeting the immune system e.g. anti-PD1 in combination with anti-CTLA-4 or anti-PDL-1 and so forth.

By “combination” is meant a composition comprising a combination of two agents or a combination which is for simultaneous, sequential or separate administration.

In one aspect, the invention provides a combination of a composition in accordance with the invention an immunotherapy, such as an immune checkpoint inhibitor or combination of immune checkpoint inhibitors. Suitably, the individual components of the combination may be administered either sequentially or simultaneously in separate or combined pharmaceutical formulations by any convenient route.

In another embodiment, where the modulator, suitably inhibitor, is for use in combination with a different cancer treatment, that cancer treatment may be a DNA damaging agent. Suitable DNA damaging agents are known to those skilled in the art.

### Assays

In another aspect, there is provided a method for screening for a modifier of a “frameshift-regulating” gene such as RNR complex gene such as RRM2, or its protein product, comprising:
a) providing a construct comprising a simple nucleotide sequence cloned upstream of a reporter coding sequence such that the reporter coding sequence is out of frame, as described in the FSR assay described herein;
b) transfecting cells with the construct of a) in combination with a construct comprising said “frameshift-regulating” gene
c) incubating said cells in the presence of a test compound;
d) measuring a signal from the reporter construct;
e) wherein an increase in signal from the reporter construct in the presence of the test compound compared to the signal in the absence of the test compound indicates that the test compound is a modifier of said “frameshift-regulating” gene.

An assay for compounds may be performed as follows: HEK293FT cells are co-transfected with WT RNR and CA₍₁₈₎-NanoLuc plasmids, cells are re-plated into 96-well plates and then treated with a dose range of potential inhibitors. Active compounds will report an increase in reporter signal, rather than an inhibition. This is a distinct advantage when dealing with immature hit compounds which may cause cellular toxicity: in assay formats which report through loss of signal, signal reduction can often be due to cell death, leading to compounds falsely being called as hits.

A test compound for use in an assay in accordance with any aspect of any embodiment of the invention may be a protein or polypeptide, polynucleotide, antibody, peptide or small molecule compound. In one embodiment, the assay may encompass screening a library of test compounds e.g. a library of proteins, polypeptides, polynucleotides, antibodies, peptides or small molecule compounds. Test compounds may also comprise nucleic acid constructs such as CRISPR constructs, siRNA molecules, anti-sense nucleic acid molecules and so forth. Suitable high throughput screening methods will be known to those skilled in the art.

Other methods for identifying a suitable test compound include the rational design of compounds. In this approach, a compound library for screening may be based on starting with those compounds known to bind to and/or inhibit/inactivate or to enhance/activate a molecule having structural similarity or homology to the DNA repair or nucleic acid editing gene of interest.

Certain aspects and embodiments of the invention will now be illustrated by way of example and with reference to the following Figures and Examples.

### FIGURES

FIG. 1. Shows a diagram to illustrate the Frameshift reporter assay. In the presence of proficient post-replicative MMR, the reporter gene remains out of frame. Inhibited or genetically deficient MMR or DNA Damage Repair can lead to frameshift mutations and an in-frame reporter which is detected using commercial NanoLuciferase assay systems. CA₂₀ is indicated here for example but can be substituted with CA₁₈, for example. The CA₂₀ tract also detects hypermutability induced by dysregulated control of cellular dNTP concentration.

FIG. 2. HEK293FT cells treated with clofarabine for 72 hours. Left: viability as determined using a CellTitre Glo assay (Promega) is shown as a fraction of DMSO control. Right: FSR activity is shown as fold increase from untreated FSR luminescence. This data shows that, when treated with clofarabine at 50 nM, a 40% increase in FSR signal is observed whilst cells remain 92% viable. The GI₅₀ for clofarabine was previously determined as 430 nM, indicating clofarabine induces frameshifts at lower concentrations comparable to GI₅₀. Results shown in triplicate, error bars indicate SD.

FIG. 3. HEK293FT cells treated with gemcitabine for 72 hours. Left: viability as determined using a CellTitre Glo assay (Promega) is shown as a fraction of DMSO control. Right: FSR luminescence as fold increase from untreated FSR luminescence. The data shows that, when treated with gemcitabine at 370 nM, a 300% increase in FSR signal is observed but also show a 50% reduction in viability. GI₅₀ for gemcitabine was determined as 440 nM, indicating gemcitabine induces frameshifts at lower concentrations comparable to GI₅₀. Results shown in triplicate, error bars indicate SD.

FIG. 4. HEK293FT cells treated with triapine for 72 hours. Left: viability as determined using a CellTitre Glo assay (Promega) is shown as a fraction of DMSO control. Right: FSR luminescence as fold increase from untreated FSR luminescence. The data shows that, when treated with triapine at 370 nM, a 400% increase in FSR signal is observed but also show a 23% reduction in viability. GI₅₀ for tripaine was determined as 600 nM, indicating triapine induces frameshifts at lower concentrations comparable to GI₅₀. Results shown in triplicate, error bars indicate SD.

FIG. 5. HEK293FT cells treated with cladribine for 72 hours. Left: viability as determined using a CellTitre Glo assay (Promega) is shown as a fraction of DMSO control. Right: FSR luminescence as fold increase from untreated FSR luminescence. The data shows that, when treated with clardribine at 3.33 uM, a 226% increase in FSR signal is observed with no reduction in viability indicating cladribine induces frameshifts at non-toxic concentrations. Results shown in triplicate, error bars indicate SD.

FIG. 6. CT26 cells treated with 100 nM clofarabine or with DMSO. Barplot showing the proportion of InDels determined for clofarabine treated and matched control samples. The proportion of InDels is defined as the total InDel count divided by the sum of the InDel and SNV counts obtained for each sample, respectively.

FIG. 7. CT26 cells treated with 100 nM clofarabine or with DMSO. Combination plot showing the number of frameshift mutations and the number of mutations (SNVs, InDel, and MNPs) per megabase (Mb) per sample.

FIG. 8. T cell killing assay using CT26 cells incubated with/without clofarabine (100 nM for 100 days). FIG. 8A. Using T cells from C56BL/6—a different strain of mice than CT26 cells. FIG. 8B. Using cells derived from Balb/c—the same strain as the CT26 cells.

### EXAMPLES

### Example 1—siRNA Screen in Frameshift Reporter (FSR) Assay

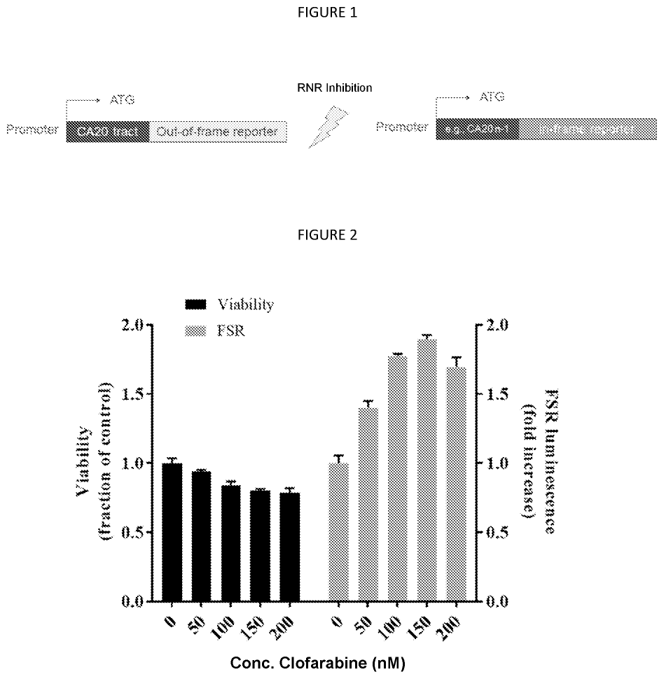

In order to read out mismatch repair (MMR) and hypermutability in mammalian cells, a Frameshift reporter (FSR) assay was used. The assay specifically focuses on frameshifts because, of all mismatch mutations, these are the ones that most abundantly generate neoantigens (see, for example, Turajlic et al. Lancet Oncol. 2017 (18; 1009-21). The assay based on the activity of the NanoLuciferase (NanoLuc®, Promega) reporter enzyme was developed (the “CA₍ₙ₎-NanoLuc assay”). Copies of the CA dinucleotide repeat (referred to as “CA₍ₙ₎”) were cloned upstream of the NanoLuc coding sequence. Typically 18 copies of the CA dinucleotide repeat (CA₍₁₈₎) were used. Alternatively, the assay used 20 copies (CA₍₂₀₎). This CA₍ₙ₎ tract renders the NanoLuc coding sequence out of frame, and therefore there is no reporter enzyme expression and no activity. The CA₍ₙ₎ tract is, however, a sequence which is subject to frequent DNA replication errors, and is therefore reliant on the MMR pathway to repair any post-replicative DNA mismatches. In a MMR-competent cell, any errors are efficiently repaired, the NanoLuc coding sequence remains out of frame and thus reporter activity is low. If, however, MMR is inhibited either by a small molecule or by genetic loss of any of the MMR machinery, post-replicative errors may remain unrepaired, frameshift mutations may occur, and therefore some cells in a population will now express a functional NanoLuc protein. This is depicted in the cartoon shown in FIG. 1.

MMR inhibition can therefore be reported as an increase in NanoLuc® activity when a plasmid containing a CA repeat, such as the CA₍₁₈₎ NanoLuc construct, is transfected into cells. Since NanoLuc® is a highly processive enzyme with a large and linear dynamic range, it was predicted that only a small number of MMR errors would be required to generate a positive signal, making for a sensitive assay with a large signal to noise ratio.

In addition, it has been shown that inhibition of RNR causes altered pools of dNTP resulting in increased mutation rates. Relatively modest dNTP pool deviations induce exceptionally strong mutator phenotypes which cause saturation of the DNA mismatch repair system and lead to detection in the FSR assay system.

This assay was used in an RNAi assay to identify drug targets within mammalian cells that, if inhibited, result in an increase in frameshift mutations and are therefore postulated to stimulate neoantigen production.

Briefly, HEK293FT cells were transiently transfected with FSR plasmid (CA₍₁₈₎-NanoLuc plasmid) and incubated with siRNA (obtained from DNA Damage Response siRNA ON-TARGET plus library from GE Dharmacon). The library, provided as 0.1 nmol 96 well master plates, were reconstituted and transferred to assay plates.

After 96 hours incubation, FSR activity was measured. siRNA corresponding to Ribonucleotide-Diphosphate Reductase subunit M2 (RRM2) induced a 240% increase in FSR signal compared to a non-coding siRNA control. Thus RRM2 knockdown by RNAi was identified to drive a large, statistically significant increase in FSR activity.

RNAi sequences are as follows:

(SEQ ID NO: 1)
1. GGAGUGAUGUCAAGUCCAA
(SEQ ID NO: 2)
2. GCGAUGGCAUAGUAAAUGA
(SEQ ID NO: 3)
3. CCACGGAGCCGAAAACUAA
(SEQ ID NO: 4)
4. GAAUUGCACUCUAAUGAAG

Detailed method as follows:

### Preparation of Cell Culture

Three T175 flasks were seeded with 2×10⁶ HEK293FT cells at passage 11 and cultured in 30 ml culture media, DMEM supplemented with 10% Fetal Bovine Serum (FBS), 1% PenStrep and L-Glutamine. Flasks incubated overnight at 37° C. (5% C02, >90% humidity).

After 24 hours media was aspirated, cells were washed in 10 ml PBS and trypsinised with 3 ml TrypIE. After 5 mins incubation TrypIE was blocked with 7 ml fresh media and cell culture was moved to a 50 ml Falcon tube. Cells were centrifuged at 1000×g for 5 mins, supernatant aspirated and resuspended in 10 ml PBS. Cells were centrifuged (1000×g, 5 mins) and PBS aspirated, cells resuspended in 10 ml DMEM growth medium.

Cell density was calculated using a ThermoScientific Countess II automated cell counter: 10 μL cell culture added to 10 μL 0.4% Trypan Blue.

Cells were diluted in a 500 mL flask to 300 000 cells/ml in DMEM. 2 ml was added to each well of a six well plate. A total of eight (8) plates were seeded and incubated overnight.

Four hours prior to transfection media was aspirated and 2 ml fresh DMEM (No PenStrep) added to each well.

Plasmid stock 003: pcDNA3.1 hygro CA18-nanoluc (1.1 μg/ml) and JetPrime Buffer (Polypus Transfection: Cat #114-75) were equilibrated to room temp. 24 μg (21 μl stock) was added to 9.6 ml JetPrime Buffer in a 15 ml Falcon tube and mixed by vortex for 10 secs. 192 μl JetPrime transfection reagent was added and mixed by vortex for 10 secs. Tube was centrifuged (54×g, 30 secs) and incubated for 10 minutes at room temperature. 200 μl of JetPrime/DNA complex was added dropwise to each well of 6 well plates containing cultured HEK293FT cells. Plates were incubated for 4 hours before aspiration of media and replacement with 2 ml/well fresh DMEM (No PenStrep) followed by overnight incubation.

On the day of siRNA transfection media was aspirated and wells washed with 2 ml PBS. 500 μl TrypIE was added to each well of 6 well plates and incubated for 5 minutes before cells were dissociated, blocked in 2 ml DMEM and all cells pooled in 2×50 ml Falcon tubes. Cells were pelleted by centrifugation at 1000×g for 5 mins and washed in 20 ml PBS followed by centrifugation (1000×g, 5 mins). PBS was aspirated and cells were resuspended in 20 ml DMEM (No PenStrep). Cell density was calculated (as described above) and cells were resuspended to 1.75×10⁵ cells/ml in 180 ml DMEM.

Preparation of siRNA Library

siRNA library (Dharmacon ON-TARGETplus® SMARTpool® siRNA Library—Human DNA Damage Response, #G-106005 Lot 11137) (“NTC”) was thawed and plates centrifuged at 54×g for 5 mins. 1.9 μl from each well was transferred to prelabelled master plates. NTC is a Non-targeting Control Pool which forms a negative control pool of four siRNAs designed and microarray tested for minimal targeting of human, mouse or rat genes. ON-TARGETplus modifications reduce potential off-targets and are recommended for determination of baseline cellular responses in RNAi experiments.

5 ml 1× siRNA buffer was made from 1 ml 5× siRNA buffer (Dharmacon) and 4 ml RNAase free water. DharmaFECT Cell Culture Reagent (DCCR) and buffer were equilibrated to room temp. 275 μl DharmaFECT 1 transfection reagent was added to 56.73 ml DCCR and mixed.

siRNA plate controls were prepared by diluting 15 μl of 20 μM siRNA stock in 15 μl 1× siRNA buffer and mixed. 1.9 μl of each stock was added to control wells in masterplates. To each well containing siRNA 93.75 μl DharmaFECT complex was added and mixed by trituration and incubated for 30 mins at room temp. 18.75 μl of each siRNA/DharmaFECT solution was sub-aliquoted into 4× prelabelled 96 well assay plates.

To each assay well 56.25 μl cell culture was added and mixed by pipetting.

### MNNG Control

A 30 mM solution of MNNG was prepared in DMEM media, 0.12 μL was added to 24.8 μL DMEM for each well. 25 μL was added to control wells containing cells to a total concentration of 12 μM.

Plates were incubated at 37° C. for 4 hours before addition of 50 μl DMEM (FBS, PenStrep) and returned to the incubator for a further 92 hours.

### Read-Out

A solution of 110 ml Nanoglo (Promega) reagent was made by addition of 2.2 ml NanoGlo substrate (Promega) to 107.8 ml Nanoglo buffer (Promega). Plates were read in batches of 8 to reduce time differences. 75 μl Nanoglo reagent was added to each well of assay plates 1 to 3 for each master plate and incubated on a plate shaker for 5 mins. 120 μl from each well was transferred to white walled, white bottom, 96 well plates and luminescence read by plate reader (BMG, ClarioStar). To plate 4 of each master plate 75 μl of CellTiter-Glo was added, plates were incubated at room temperature for 10 minutes on a plate shaker and 120 μl from each well transferred to a white walled, white bottom, 96 well plate and luminescence read by plate reader (BMG, ClarioStar).

### Example 2—a Small Molecule Inhibitor of RRM2 (RNR) Causes Frame Shift Mutations

### a) Clofarabine

Clofarabine causes a dose-dependent increase in FSR activity on HEK293FT cells. The GI₅₀ for clofarabine was previously determined as 430 nM using a standard dosimetric assay testing for cell growth.

FIG. 2 shows that an increase in FSR activity is observed even at concentrations well below those which have a cytotoxic effect. This supports the hypothesis that clofarabine might be administered at doses which are efficacious in causing increases in neoantigen number without causing extensive cytotoxicity.

### Detailed Method:

Validation of Frameshift Induction Through Inhibition of RRM2 (RNR) with Clofarabine

HEK293FT cells seeded at 600,000 cells per well in a 6 well plate and incubated overnight. Media was replaced and incubated a further 2 hours before transient transfection of pcDNA3.1 Hygro CA18Nluc (0.5 μg). After 4 hours media was replaced and incubated overnight. Media was aspirated, cells washed with PBS and trypsinised. Cells were pelleted by centrifugation (1000×g, 5 mins), washed in PBS, centrifuged again and resuspended in fresh media to 1×10≡cells/ml.

A 96 well plate was seeded with 100 μl of transfected cells and incubated overnight. A 2× solution of clofarabine was made to 100 μM in DMEM and 9× two-fold dilutions were made in growth media. A parallel dilution series was made with equal concentrations of DMSO as a control. 100 μL of clofarabine or DMSO solution was added to wells of 96 well plates (6 replicates per concentration). PBS was added to outer wells of 96 well plates and incubated for 48 hours. To 3 wells per concentration a 1:1 ratio of CellTiter Glo (Promega) was added. To remaining wells, an equal volume of NanoGlo (Promega) reagent was added. 100 μL was aliquoted from each well to a 96 well, white walled, white bottomed plate and luminescence read. Percentage viability was calculated for all conditions in comparison to an untreated control and nanoluciferase reading were normalised to viability.

### b) Gemcitabine

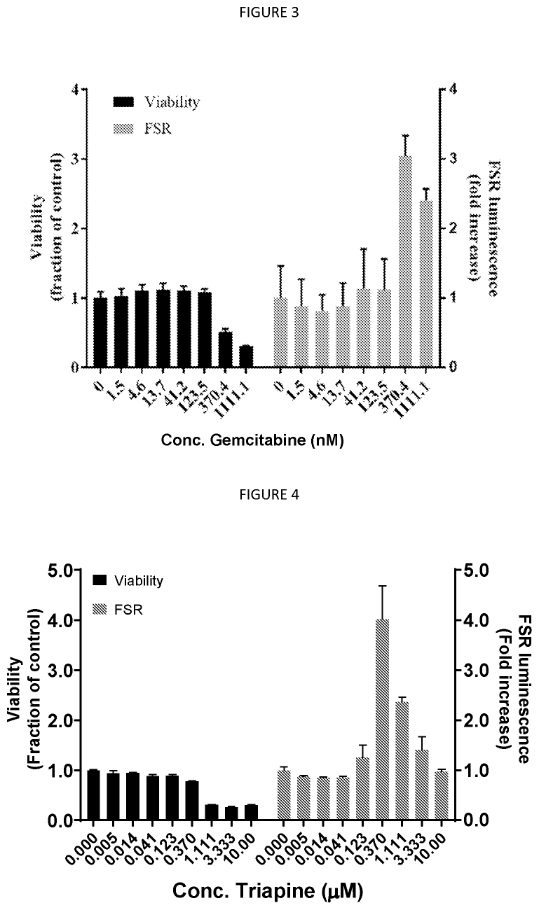

Other clinical compounds which inhibit clofarabine's target have also been tested. FIG. 3 shows results of an additional compound in the FSR assay, gemcitabine. Gemcitabine causes an increase in FSR activity, but its activity in the FSR coincides closely with its cytotoxic concentration (FIG. 3).

### c) Triapine

For triapine treatment, cells were initially seeded into 6 well plates and transfected with the pcDNA3.1 Hygro CA18Nluc construct, as in the detailed method for clofarabine treatment. After transfection, cells were trypsinised and seeded into white-walled 384 well plates at a density of 2000 cells per well in a final volume of 30 uL. After 24 hours, cells were treated with compounds or a DMSO solvent control in a final volume of 3.3 uL. Compound stock was diluted to achieve a final concentration of 10 uM, and compounds were serially diluted in DMSO by three-fold to generate the dose-response curve. Compound was subsequently diluted in media, and then added to cells in 3.3 uL, ensuring a final concentration of DMSO no greater than 0.5%. PBS was added to outer wells of the 384 well plates and incubated for 72 hours. To 3 wells per concentration, 5 uL of CellTiter Glo (Promega) was added. To remaining wells, 30 uL of NanoGlo (Promega) reagent was added and incubated at room temperature for 30 minutes. Luminescence was read. Percentage viability was calculated for all conditions in comparison to an untreated control and nanoluciferase reading were normalised to viability.

HEK293FT cells were treated with triapine for 72 hours. FIG. 4 shows that when treated with triapine at 370 nM, a 400% increase in FSR signal is observed but also show a 23% reduction in viability. GI₅₀ for tripaine was determined as 600 nM, indicating triapine induces frameshifts at lower concentrations comparable to GI₅₀.

### d) Cladribine

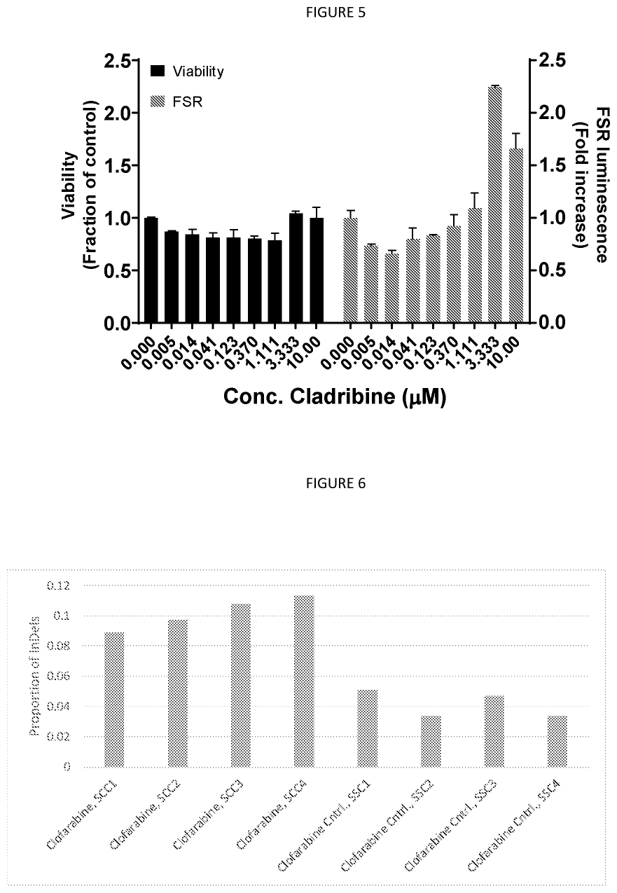

HEK293FT cells were treated with cladribine for 72 hours using the same methodology as the triapine treated cells. FIG. 5 shows that when treated with cladribine at 3.33 uM, a 226% increase in FSR signal is observed with no reduction in viability indicating cladribine induces frameshifts at non-toxic concentrations.

### Example 3—In Vivo Treatment with Clofarabine Increases Endogenous Mutation Rates

a) A Cell Line is Treated with a Sub-Cytotoxic Dose of Clofarabine Over a Period of 30 Days and Ngs Used to Detect an Increased Rate of Mutation and Thereby Neoantigen Generation, as Compared to a Vehicle Only Control.

### Detailed Method:

CT26 cells, cultured in RPM11640 are dosed with 100 nM clofarabine. Cells are passaged and re-dosed every 72 hours. Cells are harvested at 30 days, genomic DNA extracted and NGS exome sequencing performed following methods such as those described, for example, in WO2017/182783, see page 33, line 21 onwards and page 34, lines 1 to 5.

An NGS data analysis pipeline is used to identify single nucleotide variants (SNVs) and indels (small insertions and deletions). The mutational burden (number of variants) is calculated and the functional effects of the identified variants on gene transcripts predicted by examining the sequence context in which the variant occurred.

### b) Improved Clearance of Xenogenised Tumours in a Mouse Model Under ICR Treatment.

Cells previously exposed to clofarabine and expressing sufficient neoantigens or, for example, expressing an increased neoantigen repertoire, are implanted into syngeneic mice and treated with ICR. Suitable methods are described, for example, in WO2017/182783 or Germano et al. Nature 2017, 552(7683):116-120.

### Example 4—In Vivo Treatment with Clofarabine for 100 Days Increases Endogenous Mutation Rates

a) A Cell Line was Treated with a Sub-Cytotoxic Dose of Clofarabine Over a Period of 100 Days and NGS Used to Detect an Increased Rate of Mutation, and Thereby Neoantigen Generation, as Compared to a Vehicle Only Control.

### Detailed Method:

CT26 cells, cultured in RPM11640 were dosed with 100 nM clofarabine or with DMSO only. Cells were passaged and re-dosed every 72 hours. Cells were harvested at 100 days, and single-cell colonies (SSCs) derived for each sample. After expansion over 8-10 days, genomic DNA was extracted from SSC pellets and whole exome sequencing (WES) performed following methods such as those described, for example, in WO2017/182783, see page 33, line 21 onwards and page 34, lines 1 to 5.

Somatic single nucleotide variants (SNVs), multiple-nucleotide polymorphisms (MNPs), and insertion-deletion (InDel) mutations were identified using the GATK4 Toolkit and GATK's Mutect2 (version 4.1.4.0) to analyse the WES data. Prior to mapping the read sequences to the reference genome (mm10), read data quality and adapter trimming and QC was performed using TrimGalore and a Q-score cut-off of 30. Reads were aligned to mm10 using BWA mem (version 0.7.17-r1188). Duplicate reads were marked using MarkDuplicates (Picard, GATK4 copy). Variant discovery using Mutect2 was performed using a custom generated interval list and Panel of Normals (PON) resource. The interval list was derived for a BED file of canonical transcript exon positional information for mm10, which was generated using the UCSC TableBrowser. The PON was derived for matched CT26 WES data using Mutect2, GenomicsDBImport, and CreateSomaticPanelOfNormals. Variant filtering was performed using FilterMutectCalls (GATK4). Variants detailed in the VCF files generated using FilterMutectCalls were further filtered using a shell script in order to keep only variants meeting the quality control thresholds applied by FilterMutectCalls and exclude variants identified for reads mapping to unlocalized and unplaced contigs. The functional effects of the identified variants (on genes, transcripts, protein sequences, and regulatory regions) were predicted using the Ensembl Variant Effect Predictor (VEP).

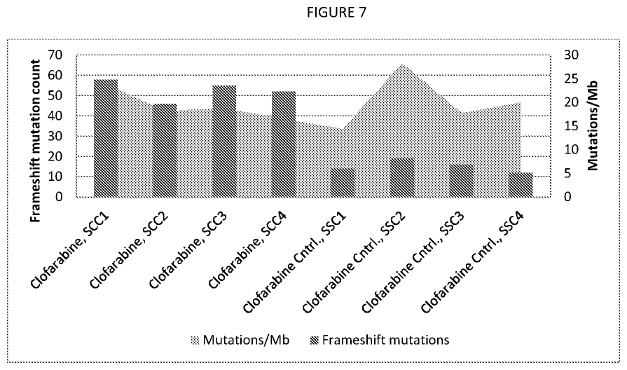

Relative to the matched vehicle only control samples, clofarabine dosed cells show an elevated total count and proportion of frame-shift causing InDel mutations (FIG. 6 and FIG. 7). This is to say that while the overall, largely SNV comprised, burden of mutations does not increase substantially with treatment, the relative proportion of InDel causing mutations does.

### b) Improved Clearance of Xenogenised Tumours in a Mouse Model Under ICR Treatment

Cells previously exposed to clofarabine and expressing sufficient neoantigens or, for example, expressing an increased neoantigen repertoire, were implanted into syngeneic mice and treated with ICR. Suitable methods are described, for example, in WO2017/182783 or Germano et al. Nature 2017, 552(7683):116-120.

### Example 5—In Vivo Treatment with Clofarabine Increases Sensitivity to T-Cell Cytotoxicity

T-cell extraction: Spleen from C56BL/6 and Balb/c mice was homogenised aseptically using a cell strainer and syringe plunger. RBC was lysed using lysis buffer and remaining cells washed and counted. Dynabeads untouched mouse CD8 cell kit (Thermofisher: 11417D) used to isolate CD8 cells using negative selection. Half these cells were cultured as unstimulated for assay controls but the majority was cultured with Dynabeads Mouse T-activator CD3/CD28 beads for 24 h prior to assay.

T-cell assay: CT26 tumour cell lines (parental as control) was plated at 3,000 cells per well into a 96 well plate; effector cells at a ratio of 10:1 was added along with appropriate stains and imaged on the EVOS microscope over a period of 72 h.

Cell lines tested in assay:
Parental CT26 cell line (controls)—Both stimulated and unstimulated T-cells
Clofarabine treated cell line (100 day)—Both stimulated and unstimulated T-cells
Clofarabine 100-day control cell line—Both stimulated and unstimulated T-cells

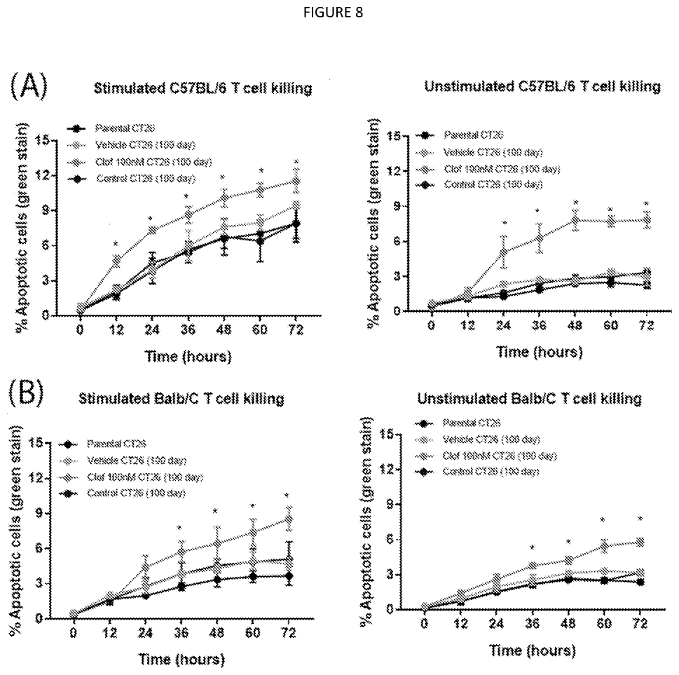

Analysis: Images was analysed using macros designed on Image J and percentage of cells staining positively for apoptosis was plotted along with tumour cell confluency. Clofarabine-treated cells significantly increase T cell killing over all other cell lines in both simulated and unstimulated, and both C57BL/6- and Balb/c-derived T cells (FIG. 8).

1. A composition for use in stimulating neoantigen production in a cancerous tumour in a subject, wherein the composition is for improving the subject's immune response against the cancerous tumour, and wherein the composition comprises a compound or a pharmaceutically acceptable salt thereof, which is a negative modulator of the expression, function or stability of ribonucleotide reductase.

2. A composition as claimed in claim 1 wherein the compound causes frameshift mutations as measured by a frameshift reporter assay.

3. A composition as claimed in claim 1 wherein the compound results in a mean luminescence greater than 3 standard deviations from a control in a frameshift reporter assay as described herein, or wherein inhibition results in a mean luminescence which is more than a 150% increase compared to a control.

4. A composition as claimed in claim 1, 2 or 3 wherein the compound has a therapeutic concentration range wherein frameshift mutations increase at a greater rate than cell viability decreases as measured by a frameshift reporter assay.

5. A composition as claimed in claim 4 which is capable of delivering a concentration of compound to the cancerous tumour, wherein the concentration of compound is within the therapeutic concentration range.

6. A composition as claimed in any preceding claim wherein the compound is an inhibitor of ribonucleotide reductase.

7. A composition as claimed in any preceding claim wherein the compound is a polypeptide, polynucleotide, antibody, aptamer, peptide, small molecule, an RNA-based drug, a genetic construct for targeted gene editing, or any other suitable chemical.

8. A composition as claimed in any preceding claim wherein the compound is a small molecule.

9. A composition as claimed in claim 8 wherein the compound is selected from clofarabine, triapine, cytarabine, cladribine, azathioprine, fludarabine, 5-flurouracil, hydroxyurea, motexafin gadolinium, gallium maltolate and gallium nitrate, or a pharmaceutically acceptable salt thereof.

10. A composition as claimed in claim 9 wherein said compound is selected from cladribine, clofarabine, cytarabine and fludarabine, or a pharmaceutically acceptable salt thereof.

11. A composition as claimed in any preceding claim wherein the cancerous tumour is microsatellite stable.

12. A composition as claimed in any preceding claim wherein the cancerous tumour is selected from neuroblastoma, glioma, glioblastoma, prostate, ovary, myeloma, pancreas, breast, papillary kidney, B cell lymphoma, clear cell kidney, head and neck, liver, cervix, uterus, bladder, colorectal, small cell lung, espohagus, stomach, non-small cell lung cancer, and melanoma.

13. A composition as claimed in claim 12 wherein the cancerous tumour is selected from head and neck, liver, cervix, uterus, bladder, colorectal, small cell lung, espohagus, stomach, non-small cell lung cancer, and melanoma.

14. A composition as claimed in claim 13 wherein the cancerous tumour is selected from head and neck, liver, cervix, uterus, bladder, colorectal, small cell lung, espohagus, and stomach.

15. A composition as claimed in any preceding claim wherein the treatment comprises administering a therapeutically effective amount of the compound to the subject.

16. A composition as claimed in any preceding claim for use in combination with an immunotherapy.

17. The composition as claimed in claim 16 wherein the immunotherapy is an immune checkpoint inhibitor or a combination of immune checkpoint inhibitors.

18. The composition as claimed in claim 16 or 17 wherein the immunotherapy comprises an immune checkpoint inhibitor selected from PD-1 inhibitor, a PD-L1 inhibitor, and a CTLA-4 inhibitor.

19. The composition as claimed in claims 16 to 18 wherein the immunotherapy comprises an immune checkpoint inhibitor selected from pembrolizumab, nivolumab, ipilimumab, atezolizumab, avelumab, durvalumab, tremelimumab, tislelizumab, pidilizumab, AMP-224, AMP-514, PDR001, BMS-936559.

20. The composition as claimed in any one of claims 16 to 19 wherein the immunotherapy is administered subsequent to or concurrent with the composition.

21. A method for improving the immune response to a cancerous tumour in a subject in need thereof, the method comprising the steps of:
a. Assessing the level of neoantigen presentation of the cancerous tumour;
b. Determining whether the level of neoantigen presentation is below a threshold to induce an immune response, and
c. If the level of neoantigen presentation is below a threshold to induce an immune response, administering a composition comprising a compound or a pharmaceutically acceptable salt thereof, which is a negative modulator of the expression, function or stability of ribonucleotide reductase.

22. A method as claimed in claim 21 wherein the level of neoantigen presentation is assessed with reference to the number of mutations in the genome of the cancerous tumour.

23. A method as claimed in claim 22 wherein the threshold to induce an immune response is 100 mutations per megabase.

24. A method as claimed in claim 21 to 23 comprising the further step of:
d. Reassessing the level of neoantigen presentation of the cancerous tumour.

25. A method as claimed in claim 21 to 24 comprising the further step of:
e. Administering an immunotherapy to the subject.

26. The method of any one of claims 21 to 25 wherein the composition is as claimed in any one of claims 1 to 20.


SEQUENCE LISTING (4 sequences)

SEQ ID NO: 1 - RNA, 19 nt, Artificial Sequence
  siRNA 1
ggagugaugucaaguccaa

SEQ ID NO: 2 - RNA, 19 nt, Artificial Sequence
  siRNA 2
gcgauggcauaguaaauga

SEQ ID NO: 3 - RNA, 19 nt, Artificial Sequence
  siRNA 3
ccacggagccgaaaacuaa

SEQ ID NO: 4 - RNA, 19 nt, Artificial Sequence
  siRNA 4
gaauugcacucuaaugaag